(19) United States
(12) Patent Application Publication
(10) Pub. No.: US 2020/0384020 A1
(43) Pub. Date: Dec. 10, 2020

(54) IMMUNE CELLS WITH MODIFIED METABOLISM AND THEIR USE THEREOF

(71) Applicant: TC BIOPHARM LTD, Holytown Strathclyde (GB)

(72) Inventors: TIMOTHY LONDON, Glasgow Strathclyde (GB); AGAPITOS PATAKAS, Glasgow Strathclyde (GB); ADELE HANNIGAN, Newlands, Glasgow Strathclyde (GB); EMILIO COSIMO, Muirhead, Glasgow Strathclyde (GB); NANCY COYLE, Holytown, Motherwell Strathclyde (GB); ANGELA SCOTT, Edinburgh Lothian (GB); MICHAEL LEEK, Edinburgh Lothian (GB)

(21) Appl. No.: 16/463,961

(22) PCT Filed: Jan. 26, 2018
(86) PCT No.: PCT/GB2018/050240

§ 371 Date: May 24, 2019

(30) Foreign Application Priority Data
Jan. 26, 2017 (GB) 1701332.7

(51) Int. Cl. A61K 35/17; C12N 5/0783; C07K 14/705

(52) U.S. Cl. CPC A61K 35/17; C07K 14/705; C12N 5/0636

(57) ABSTRACT

A modified T cell is described which is adapted to overexpress SLC1A5, an isoform of SLC1A5 or an alternative tryptophan or glutamine transporter. Further described is the use of such modified T cells in the treatment of disease, in particular cancer, methods to select modified T cells which overexpress SLC1A5 and nucleic acids and vectors to provide for such modified T cells.

The present invention relates to T cells adapted to function in a low tryptophan or tryptophan depleted micro-environment, in particular an environment in which tryptophan catabolism occurs, wherein the T cells have been modified such that they express amino acid transporters, suitably glutamine and/or tryptophan transporters, for example SLC1A5 and its isoforms. The present invention further provides methods to provide such T cells and uses thereof.

### BACKGROUND OF THE INVENTION

Tryptophan degradation is an immune escape strategy which is utilised by many tumours.

Indoleamine 2,3-dioxygenase (IDO) and tryptophan 2,3-dioxgenase (TDO) are rate limiting enzymes of the kynurenine pathway, which convert the essential amino acid tryptophan to kynurenine.

Low tryptophan conditions, typically <5 μM, caused by IDO activity induces extensive remodelling of tumour cells, amino acid metabolism, gene expression and also the upregulation of expression of amino acid transporter encoding genes such as SLC7A11, SLC1A4 and SLC1A5, including SLC1A5 splice variants.

SLC1A5 is a sodium-dependent high-affinity glutamine transporter of the solute carrier family. Upregulation of the expression of SLC1A5 (and its splice variants) improves the uptake of glutamine into tumour cells. In addition to enhancing the uptake of glutamine, upregulation of expression of SLC1A5 also improves tryptophan transport by enhancing the activity of the large neutral amino acid transporter (LAT1). LAT1 is a heterodimeric membrane transport protein that preferentially transports branched-chain (valine, leucine, isoleucine) and aromatic (tryptophan, tyrosine) amino acids. A functional LAT1 transporter is composed of two proteins encoded by two distinct genes:
1. 4F2hc/CD98 heavy subunit protein encoded by the SLC3A2 gene, and
2. CD98 light subunit protein encoded by the SLC7A5 gene.

Being an obligate amino acid exchanger, LAT1 activity depends largely on the exchange of intracellular glutamine for the uptake of branched chain and aromatic amino acids.

Constitutive expression of IDO and TDO has been reported in a number of human cancers leading to tryptophan catabolism in the tumour microenvironment. Limited tryptophan availability has profound immunoregulatory effects leading to reduced proliferation and effector functions of T cells. Cancer cells are protected by this hostile microenvironment by upregulation of amino acid transporters that give the cancer cells a selective advantage over other cells in the tumour.

Whilst it has been established that tryptophan catabolism has immunosuppressive effects on T cells, the mechanism by which tryptophan catabolism influences T cells is less understood.

IDO has been the focus of attention in recent years because of its immunosuppressive effects on T lymphocytes, resulting partly from tryptophan depletion and partly from direct effects of tryptophan catabolites.

TDO, the tryptophan degrading enzyme, has been observed to provide immunosuppressive effects.

TDO and IDO inhibitors have been suggested to promote tumoral immune rejection and to improve the efficiency of cancer immunotherapy.

### SUMMARY OF THE INVENTION

IDO is a cytosolic enzyme, therefore tryptophan degradation by IDO occurs inside the cell. However, as tryptophan readily crosses the plasma membrane through specific transporters, the cells act as tryptophan sinks causing the microenviroment around the tumour cells to be low in tryptophan. Tryptophan catabolism mediated by an indolamine 2,3-dioxygenase (IDO) is an important mechanism of peripheral immune tolerance contributing to tumoural immune evasion due to tryptophan depletion in the tumour micro-environment. It would be beneficial if T cells could be provided, which have an ability to target cancer cells and have the ability to resist the immunoregulatory tumour micro-environment in which tryptophan catabolism occurs.

The inventors have determined a method by which T cells can be provided with resistance to proliferative arrest following exposure to low tryptophan conditions, in particular as caused by a tumour expressing IDO or TDO enzyme(s), the method comprising providing a T cell over-expressing SLC1A5, an isoform of SLC1A5 or an alternative tryptophan or glutamine transporter.

T-cells are divided into two groups based on their T-Cell Receptor (TCR) components. The TCR heterodimer can include an α and β chain. An α and β TCR recognises foreign antigens via peptides presented by MHC molecules on antigen presenting cells. The TCR heterodimer can alternatively include a γ and δ chain. TCRs including γ and δ chains, (γδ TCRs) are MHC independent.

Full activation of a T cell which results in the effective killing of a target cell requires productive signal 1 and signal 2 generation. Having received signal 1 from the TCR/CD3 signal, signal 2 is provided by co-stimulatory molecules, for example CD28.

Suitably a T cell may be considered to be a cell which expresses an αβ TCR or a γδ TCR. Suitably, the T cell may be a gamma delta (γδ) T cell which expresses a TCR of any gamma delta TCR pairing from Vgamma(γ)1 to 9 and Vdelta(δ)1 to 8 The γδ T cell may be of the Vγ9Vδ2 subtype.

Accordingly, a first aspect of the present invention provides a T cell over-expressing SLC1A5, an isoform of SLC1A5 or an alternative tryptophan or glutamine transporter. Alternative transporters may include other members of the high-affinity glutamate and neutral amino acid transporter family (SLC1A1, SLC1A2, SLC1A3, SLC1A4, SLC1A5, SLC1A6, SLC1A7), the heavy subunits of heterodimeric amino acid transporters (SLC3A1, SLC3A2), members of the sodium- and chloride-dependent sodium:neurotransmitter symporter family (SLC6A1, SLC6A2, SLC6A3, SLC6A4, SLC6A5, SLC6A6, SLC6A7, SLC6A8, SLC6A9, SLC6A10, SLC6A11, SLC6A12, SLC6A13, SLC6A14, SLC6A15, SLC6A16, SLC6A17, SLC6A18, SLC6A19, SLC6A20) or members of cationic amino acid transporter/glycoprotein-associated family (SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14).

As would be understood by those of skill in the art, for example as discussed in Timosenko et al, “Nutritional Stress induced by tryptophan-degrading enzymes results in ATF4-dependent reprogramming of the amino acid transporter profile in tumor cells”, Cancer Res. 2016 76 (21):6193-6204, SLC1A5 is known to exist as a full length transcript (SLC1A5 long (SLC1A5-L)) and as truncated splice variants, including SLC1A5 middle (SLC1A5-M) and SLC1A5 short (SLC1A5-S)).

Suitably the T cell may express SLC1A5, an isoform of SLC1A5 or a tryptophan or glutamine transporter, optionally wherein the transporter is selected from a high-affinity glutamate and neutral amino acid transporter family (SLC1A1, SLC1A2, SLC1A3, SLC1A4, SLC1A5, SLC1A6, SLC1A7), heavy subunits of heterodimeric amino acid transporters (SLC3A1, SLC3A2); a member of the sodium- and chloride-dependent sodium:neurotransmitter symporter family (SLC6A1, SLC6A2, SLC6A3, SLC6A4, SLC6A5, SLC6A6, SLC6A7, SLC6A8, SLC6A9, SLC6A10, SLC6A11, SLC6A12, SLC6A13, SLC6A14, SLC6A15, SLC6A16, SLC6A17, SLC6A18, SLC6A19, SLC6A20) or a member of a cationic amino acid transporter/glycoprotein-associated family (SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14) at a level at least two, at least three, at least four, at least five, at least six, at least seven, at least eight, at least nine, at least ten, at least 20, at least 50, at least 100 times the expression level as typically observed in a T cell. Expression levels of endogenous SLC1A5 or alternative tryptophan or glutamine transporters in unmodified T cells may be determined using techniques such as western blotting or flow cytometry and compared to the levels in genetically modified T cells.

Suitably the T cell may express SLC1A5, an isoform of SLC1A5 or a tryptophan or glutamine transporter, optionally wherein the transporter is selected from a high-affinity glutamate and neutral amino acid transporter family (SLC1A1, SLC1A2, SLC1A3, SLC1A4, SLC1A5, SLC1A6, SLC1A7), heavy subunits of heterodimeric amino acid transporters (SLC3A1, SLC3A2); a member of the sodium- and chloride-dependent sodium:neurotransmitter symporter family (SLC6A1, SLC6A2, SLC6A3, SLC6A4, SLC6A5, SLC6A6, SLC6A7, SLC6A8, SLC6A9, SLC6A10, SLC6A11, SLC6A12, SLC6A13, SLC6A14, SLC6A15, SLC6A16, SLC6A17, SLC6A18, SLC6A19, SLC6A20) or a member of a cationic amino acid transporter/glycoprotein-associated family (SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14) at a level at least two, at least three, at least four, at least five, at least six, at least seven, at least eight, at least nine, at least ten, at least 20, at least 50, at least 100 times the expression level as typically observed in an activated T cell.

Suitably, the T cell may be a gamma delta T cell. In embodiments the gamma delta T cells may be activated (i.e. when they proliferate more rapidly and secrete cytokines). The T cell may be an alpha beta T cell. The alpha beta T cell may be activated. The T cell may be a gamma delta or alpha beta T cell comprising an SLC1A5 transporter or an isoform thereof and/or a glutamine or tryptophan transporter together with a chimeric antigen receptor (CAR) capable of binding to tumour antigen. Suitably, the CAR may be a CAR providing a signal 1 response only, for example from a CD3zeta domain, or a signal 1 and a signal 2 response from for example a CD3zeta domain and a co-stimulatory domain, when the extracellular portion of the CAR binds to an antigen. Such CARs may be useful for use with alpha beta T cells. The CAR may be a co-stimulatory CAR and only provide a signal 2 response on antigen binding as discussed by WO2016/166544. A CAR which provides only a signal 2 response via, for example, a co-stimulatory domain may be advantageous for use with gamma delta T cells wherein a signal 1 may be provided by binding of the T cell receptor (TCR) on the gamma delta T cell to the antigen recognised by the TCR.

Suitably the T cell may be an alpha beta T cell or a gamma delta T cell which over-expresses SLC1A5, or an isoform thereof and/or a glutamine or tryptophan transporter together with a chimeric antigen receptor (CAR) which is capable of binding specifically to a disease antigen.

Suitably, the T cell may be a gamma delta (γδ) T cell which expresses a TCR of any gamma delta TCR pairing from Vgamma(γ)1 to 9 and Vdelta(δ)1 to 8 and which expresses SLC1A5, or an isoform thereof and/or a glutamine or tryptophan transporter together with a chimeric antigen receptor (CAR) which is capable of binding specifically to a disease antigen. The γδ T cell may be of the Vγ9Vδ2 subtype.

Gamma delta T cells may comprise a glutamine and/or tryptophan transporter such as SLC1A5 and a CAR. Suitably the CAR may be a classical or non-tuneable CAR (a CAR which can provide signal 1 and signal 2). A classical CAR comprised of an extracellular antigen binding domain, a hinge region, a transmembrane domain, one or more co-stimulatory domains (providing signal 2) and a signal 1 providing activation domain e.g. CD3zeta. In embodiments, the CAR may be a co-stimulatory CAR including only co-stimulatory domains, but not including a signal 1 providing activation domain (such that upon binding to the CAR only a costimulatory signal is provided (signal 2) (i.e. no signal 1 is provided through activation of the costimulatory-CAR alone)). In such embodiments, a second receptor present on the T cell, such as a T cell receptor (TCR), may provide signal 1 to allow the signal 1 and signal 2 to synergise to permit activation of the T cell.

SLC1A5 may be overexpressed alone, or in conjunction with SLC7A5 and SLC3A2 to form the LAT1 transporter, further upregulating the uptake of tryptophan by the T cell.

Suitably the T cell may express SLC1A5 and/or the glutamine or tryptophan transporter at a level at least two, at least three, at least four, at least five, at least six, at least seven, at least eight, at least nine, at least ten, at least 20, at least 50, at least 100 times the expression level as typically observed in a T cell.

Suitably the T cell may express SLC1A5 and/or the glutamine or tryptophan transporter at a level at least two, at least three, at least four, at least five, at least six, at least seven, at least eight, at least nine, at least ten, at least 20, at least 50, at least 100 times the expression level as typically observed in an activated T cell. Over expression may be effected by any means known in the art. Suitably over expression functionally allows a modified T cell to advantageously function in a low tryptophan micro-environment, for example in a low tryptophan micro-environment as detected around some tumour cells.

A modified γδ T cell adapted to function in a low tryptophan micro-environment in which tryptophan catabolism occurs may comprise a chimeric antigen receptor wherein the chimeric antigen receptor comprises an extracellular antigen binding domain with binding specificity to a disease antigen, a transmembrane domain, and

(i) at least one co-stimulatory signalling region (able to provide signal 2, but not signal 1) and no signal 1 providing signalling domain, for example CD3zeta (to provide a ‘co-stimulatory’ or ‘tuneable’ CAR), or

(ii) a CD3zeta activation/signalling domain (able to provide signal 1), or

(iii) at least one co-stimulatory signalling region and a CD3zeta (classical CAR able to provide signal 1 and signal 2) activation/signalling domain.

Suitably, when the nucleic acid sequence of the CAR includes the CD3zeta domain, the CAR is considered ‘classical’ or ‘non-tuneable’. In embodiments in which the CAR contains only co-stimulatory domains it can be considered a ‘co-stimulatory’ or ‘TCR-tuneable’ CAR.

A nucleic acid sequence encoding the CAR, ‘classical’ or ‘co-stimulatory’ may comprise a single chain variable fragment (scFv) recognising a disease-associated antigen or tumour antigen or protein or carbohydrate or lipid or small molecule.

The antigen binding domain of the CAR may take many forms, including (but not limited to), a single chain variable fragment (scFv) derived from an antibody, a nanobody, a growth factor sequence, a synthetic sequence based on a soluble factor, a sequence based on a factor which binds to a receptor ecto-domain, or the extracellular domain of a cell surface receptor which is then fused to the transmembrane and co-stimulatory domains as described above.

Suitably the disease antigen may be a viral antigen.

The disease antigen may be a cell surface target or an antigen found in a tumour, a cell infection, bacterial infection, fungal infection or protozoan infection or can be an active or inactivated viral fragment, a peptide, a protein, an antigenic segment or the like from such a virus. The cell surface target may include a tumour-specific antigen and/or tumour associated antigen.

Suitably, the extracellular antigen binding domain may recognise and bind to a tumour-specific or disease-associated antigen which is present only on tumour/diseased cells and not on any other cells and/or a disease-associated antigen which is present on some diseased cells and also some normal cells. Such disease associated antigens may include, but are not limited to, CD19, EGFR, EGFRvRIII, ErbB2, GM3, GD2, GD3, CD20, CD22, CD30, CD37, CD38, CD70, CD75, CD79b, CD33, CD138, gp100, NY-ESO-1, MICA, MICB, MART1, AFP, ROR1, ROR2, PSMA, PSCA, mutated Ras, p53, B-Raf, c-met, VEGF, carbonic anhydrase IX, WT1, carcinoembryonic antigen, CA-125, MUC-1, MUC-3, epithelial tumour antigen and a MAGE-type antigen including MAGEA1, MAGEA3, MAGEA4, MAGEA12, MAGEC2, BAGE, GAGE, XAGE1B, CTAG2, CTAG1, SSX2, or LAGE1 or viral antigens or combinations thereof or post-translationally modified proteins that may include, but are not limited to, carbamylated and citrunillated proteins.

The cell surface antigen can be an immune checkpoint ligand, for example PD-L1 or PD-L2.

The transmembrane domain of a CAR can comprise one or more of the transmembrane domains of CD3 or CD4 or CD8 or CD28 or parts thereof.

The costimulatory signalling region of the CAR may comprise for example one or more of the signal 2-providing intracellular domains of CD28, CD137 (4-1BB), ICOS, CD27, OX40, LFA1, PD-1, CD150, CD154, CD244, NKG2D, DNAX-Activating protein (DAP)-10, DAP-12, LIGHT, Fc receptor γ chain, IL-2 common γ chain, IL-12 receptor.

According to a second aspect of the invention there is provided a method of treating a cancer, suitably a cancer in a mammal, preferably a human, the method comprising administration of an effective amount of a T cell of the first aspect of the invention.

According to a third aspect of the invention there is provided an isolated nucleic acid encoding SLC1A5, an isoform of SLC1A5 or a tryptophan or glutamine transporter operably linked to control sequences adapted to allow a T cell transformed by the nucleic acid to be capable of expressing the encoded tryptophan or glutamine transporter, for example SLC1A5.

The nucleic acid sequence for the expression of the SLC1A5, an isoform of SLC1A5 or a tryptophan or glutamine transporter may comprise the following elements;
a promoter for example, but not limited to, CMV, EF1α, MSCV, PGK, CAG, IRES or UBC
the nucleic acid sequence of SLC1A5, an isoform of SLC1A5 or a tryptophan or glutamine transporter suitably including an N-terminal Kozak sequence
a RNA splice/polyadenylation sequence for example, but not limited to BGH or SV40.

In embodiments wherein the T cell comprises a CAR, the SLC1A5 sequence, an isoform of SLC1A5 or a tryptophan or glutamine transporter may be operably linked to a separate promoter from that of the CAR to produce two independent mRNAs. Suitably, expression of the CAR and the transporter encoded by the transporter encoding nucleic acid sequence may be achieved by transcription from a common, bi-directional promoter to produce two independent mRNAs. Alternatively, expression of the CAR and the transporter sequence may be achieved by transcription from a single promoter and by incorporation of an internal ribosomal entry site (IRES) between the two coding sequences to produce a single mRNA capable of translating two proteins. Suitably, the CAR and transporter sequence may be separated by a self-cleaving T2A cleavage sequence providing a single mRNA, driven from a common promoter, translating a single polypeptide which will be co-translationally cleaved to generate two proteins.

According to a fourth aspect of the present invention there is provided a vector comprising a nucleic acid of the third aspect of the invention.

Any suitable vector to introduce nucleic acid which may allow over expression of nucleic acid sequence of SLC1A5, an isoform of SLC1A5 or a tryptophan or glutamine transporter may be used. The vector backbone may contain a bacterial origin of replication such as, for example, pBR322 and a selectable marker conferring resistance to an antibiotic, such as, but not limited to, the beta-lactamase gene conferring resistance to the antibiotic ampicillin to allow for sufficient propagation of the plasmid DNA in a bacterial host. Optionally, the vector may include the bacterial and phage attachment sites (attB and attP) of an integrase such as phiC31 in combination with the recognition sites of an endonuclease such as I-Scel to allow the production of minicircles devoid of the bacterial backbone. The vector will also include a sequence which encodes for expression of SLC1A5, or an isoform thereof, or an alternative tryptophan or glutamine transporter linked to a suitable promoter sequence for expression in the target cell of interest, most preferably a T cell. Optionally, the vector may include an antibiotic resistance gene, for positive selection in mammalian cells and may also include a reporter gene for identification of expression such as, but not limited to, green fluorescence protein (GFP). Additional reporter and/or selection gene expression may be driven from individual promoters, a bi-directional promoter or achieved by use of an IRES or self-cleaving T2A sequence.

According to a fifth aspect of the present invention there is provided a host T cell transformed with the nucleic acid of the third aspect or vector of the fourth aspect of the present invention.

The method of genetically modifying a T cell to incorporate the nucleic acid encoding SLC1A5 or an alternative tryptophan or glutamine transporter may include any technique known to those skilled in the art.

Suitable methodologies include, but are not restricted to, viral transduction with viruses e.g. lentiviruses/retroviruses/adenoviruses, cellular transfection of nucleic acids by electroporation, nucleofection, lipid-based transfection reagents, nanoparticles, calcium chloride based transfection methods or bacterially-derived transposons, DNA transposons or retrotransposons, TALENS or CRISPR/Cas9 technologies.

Suitably, the genetic information provided to modify the T cell may take the form of DNA (cDNA, plasmid, linear, episomal, minicircle), RNA or in vitro transcribed (IVT) RNA. In addition to the genetic information encoding the transporter(s) and/or the CAR sequences, the genetic information may also encode for proteins/enzymes/sequences required to aid integration of the genetic information into the host genome.

When lentiviruses/retroviruses/adenoviruses are employed for transduction, inclusion of chemical reagents as would be understood by those skilled in the art to enhance this process can be used. These include for example, but are not limited to, hexadimethrine bromide (polybrene), fibronectin, recombinant human fibronectin (such as RetroNectin-Takara Clontech), DEAF dextran and TransPlus Virus Transduction Enhancer (ALSTEM Cell Advancements).

Suitably, incorporation of nucleic acids encoding a transporter and/or a CAR may be introduced to T cells, peripheral blood mononuclear cells (PBMCs), cord blood mononuclear cells (CBMCs) or tissue derived expanded T cells at any time-point over the culturing period.

According to a sixth aspect of the present invention there is provided a method of culturing host T cells such that the nucleic acid of the third aspect or the vector of the fourth aspect capable of expressing the transporter is expressed by the T cell. Optionally, an embodiment of the method of culturing a host cell further comprises recovering the T cell from the cell culture medium.

According to a further aspect of the present invention there is provided a method of delivering a T cell of the present invention to a tumour cell expressing SLC1A5, an isoform of SLC1A5 or a glutamine or tryptophan transporter wherein the micro-environment around the tumour cell is depleted of tryptophan. Suitably in embodiments the tryptophan depletion may cause at least one, at least two, at least three, at least four, at least five times less tryptophan than in a typical cellular micro-environment surrounding a cell in the host animal. To assess tryptophan depletion, the expression of a suitable transporter may be monitored using for example flow cytometry, western blotting, immunocytochemistry, qPCR or the like and combinations thereof.

According to a further aspect of the present invention there is provided a composition comprising a T cell of the present invention together with a therapeutic agent, suitably an anti-cancer agent.

Suitably, the therapeutic agent may be selected from the group consisting of a radionucleotide, boron, gadolinium or uranium atoms, an immunomodulator, an immunoconjugate, a cytokine, a hormone, a hormone agonist, an enzyme, an enzyme inhibitor, a photoactive therapeutic agent, a cytotoxic drug, a toxin, an angiogenesis inhibitor, immune-checkpoint inhibitor, a therapeutic antibody, antibody-drug conjugate (ADC) or a combination thereof.

The therapeutic agent may comprise an immunoconjugate/ADC comprising a cytotoxic drug. Suitably the cytotoxic drug may be a drug, a prodrug, an enzyme or a toxin.

In embodiments the method of treating a cancer in a subject, suitably a mammal, particularly a human, can comprise treating the subject with a therapeutically effective amount of a T cell of the present invention. In embodiments, the T cell may be provided in a therapeutically effective formulation of T cells in a dosage of 1×10⁴ cells per kg of body weight, to over 5×10⁸ cells per kg of body weight of the subject per dose.

In embodiments the method can comprise repeatedly administering a therapeutically effective formulation of T cells.

In embodiments the cancer to be treated can be selected from (but not limited to) renal, brain, ovarian, cervical, lung, bladder, oesophageal, colorectal, skin, melanoma, leukaemia, myeloma, lymphoma, bone, hepatocellular, endometrial, pancreatic, uterine, head and neck, salivary gland, breast, prostate or colon cancer.

As used herein, the term SLC1A5 can refer to a neutral amino acid transporter with a preference for zwitterionic amino acids. Suitably, it can accept a substrate neutral amino acid including glutamine, asparagine and branched chain and aromatic amino acids. It may also include methylated, anionic and/or cationic amino acids.

SLC1A5 can also be referred to as ASCT2 or ATBO and can function as a sodium dependent amino acid transporter.

In embodiments SLC1A5 can be R16, AAAT, NZA1, RDRC, ASCT-T and N7BS1. SLC1A5 may also be referred to as Solute Carrier Family 1 Member 5, Solute Carrier Family 1 (Neutral Amino Acid Transporter) Member 5, Sodium-Dependent Neutral Amino Acid Transporter Type 2, RD114/Simian Type D Retrovirus Receptor, Baboon M7 Virus Receptor, ATB(0), ASCT2, M7V1, RDRC, Neutral Amino Acid Transporter B(0), Neutral Amino Acid Transporter B, RD114 Virus Receptor, M7VS1, AAAT, ATBO, R16 and RDR.

A nucleic acid sequence for human SLC1A5 can be found on NIHNCBI sequence websites under accession number BC000062. In embodiments an amino acid sequence may be provided by accession number AAH00062.1.

An SLC1A5 variant may have at least 80%, at least 85%, at least 90%, at least 95%, at least 96%, at least 97%, at least 98%, at least 99% or 100% sequence identity to the sequence provided by AAH00062.1. As will be appreciated, such a variant should encode a protein that can function as a transporter, in particular to enhance tryptophan uptake into a modified T cell. As indicated, SLC1A5 exists in truncated isoforms. Accordingly, variants that are fragments of SLC1A5 which can suitably encode a protein that can function as a transporter are provided. Functional activity screening can be utilised to determine suitable N-terminal or C-terminal deletion proteins encoded by such variants of fragments of SLC1A5.

Suitably a variant of the human SLC1A5 gene may be provided by a homolog from another animal, for example mouse or rat or the like. Suitably such homologs may show a sequence homology of at least 80%, at least 85%, at least 90%, at least 95%, at least 96%, at least 97%, at least 98%, or at least 99% sequence identity.

Homology is determined as a percentage of residues in the amino acid sequence or nucleic acid sequence which are identical between the variant and SLC1A5 as discussed herein after aligning the sequences and introducing gaps, if necessary, to achieve the maximum percent homology.

Methods of, and computer programs for alignment and performing homology and sequence identity are well known in the art.

As used herein, a SLC1A5 variant can include amino acid sequence modifications of SLC1A5 wherein the variants are provided by introducing appropriate nucleotide changes into the nucleic acid encoding the SLC1A5 transporter. Modifications can include deletions, insertions, substitutions or the like. Suitably amino acid changes can be made wherein the amino acid changes include deletion, insertion and/or substitution or which alter the post-translational modification processes of the SLC1A5 transporter, for example the number and/or position of glycosylation sites thereon.

Suitably, techniques such as alanine scanning mutagenesis can be used to determine where suitable amino acid substitutions can be made. This can be used in combination with functional screening to determine where substitutions, deletions or insertions provide for appropriate functional activity of the variant polypeptides.

Variant polypeptides can also include modifications at the C or N-terminus of the polypeptide. As would be known in the art, suitably substitutions of nucleic acids encoding amino acids or of amino acids resulting in conservative substitutions, wherein similar amino acids based on common side chain properties for example hydrophobic, neutral, hydrophilic, acidic, basic, chain orientation, or aromatic residues are considered to be conserved, can be provided.

Suitably, nucleic acid molecules encoding amino acid sequence variants of a transporter can be prepared by a variety of methods known in the art. These methods can include but are not limited to preparation by site directed mutagenesis, PCR mutagenesis, cassette mutagenesis or the like.

In embodiments “therapeutically effective” refers to an amount of T cell effective to treat a disease or disorder in a mammal, in particular, cancer. A “therapeutically effective” amount in relation to T cells and cancer may be the number of T cells required to reduce the number of cancer cells, for example reduce tumour size, inhibit or slow the extent or stop cancer cell infiltration into peripheral organs, inhibit, slow or stop tumour metastasis, inhibit, slow or stop the growth of cancer and/or inhibit, slow or stop one or more symptoms associated with the cancer.

Suitably, administration of a therapeutically effective amount of T cells may prevent growth and/or kill existing cancer cells. In relation to cancer therapy, a therapeutically effective amount can, for example, be measured by assessing the time to disease progression and/or determining treatment response rates. Suitably, in addition to provision of T cells further anti-cancer treatments may be provided.

The term “cancer” as used herein refers to a physiological condition in mammals, particularly humans, characterised by unregulated cell growth.

In embodiments this can include benign, pre-cancerous, malignant, metastatic, non-metastatic cells. Examples of cancers include but are not limited to carcinoma, lymphoma, blastoma, sarcoma and leukemia or lymphoid malignancies. Suitably, cancers can include squamous cell cancers, lung cancer, including small cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary carcinoma, kidney or renal cancer, prostate cancer, vulvul cancer, thyroid cancer, hepatic carcinoma, anal carcinoma, penile carcinoma as well as head and neck cancer.

Suitably, tumour response can be assessed for changes in tumour morphology, for example in relation to tumour burden, tumour size and the like or using MRI scanning, x-ray scanning, CT scanning, bone imaging or biopsy sampling.

Herein, an isolated nucleic acid molecule may be a nucleic acid molecule that is identified and separated from at least one contaminate nucleic acid molecule with which it is ordinarily associated with the natural source of the nucleic acid. Isolated nucleic acids can also include nucleic acids which are in a different form or setting from which they are found in nature.

Isolated nucleic acid also includes a nucleic acid molecule contained in a cell that ordinarily expresses the nucleic acid, but which is provided in a different location in the cell, for example a different chromosomal location. Control sequences as used herein refers to DNA sequences for the expression of an operably linked coding sequence in a host organism. Suitably the control sequences are suitable to allow expression of an operably linked coding sequence in a T cell.

Nucleic acid that is operably linked as used herein describes a nucleic acid that is placed into a functional relationship with another nucleic acid sequence. For example, DNA for a secretory leader sequence is operably linked to DNA for a polypeptide if it is expressed as a pre-protein that allows for secretion of the polypeptide. A promoter or enhancer is operably linked to a coding sequence if it affects the transcription of the coding sequence.

A further aspect of the present invention can comprise a chemotherapeutic agent, a cytotoxic agent, a cytokine, a growth inhibitory agent, an anti-hormonal agent, anti-angiogenic agent and a T cell of the present invention, forming a composition, such that the components of the composition are provided simultaneously, sequentially or separately in combination with the amounts effective for the purpose intended.

In embodiments a composition of the present invention can be provided following testing of the subject or a tumour cell obtained from a subject to determine if the tumour cell has a tryptophan depleted micro-environment around the cell.

Accordingly there is provided a method to treat tumours expressing IDO or TDO comprising the steps of
providing a T cell or composition of the present invention to a subject with a tumour cell expressing an elevated level of IDO or TDO,
optionally the method can comprise the step of detecting the presence of elevated expression of IDO or TDO in a tumour cell.

According to a further aspect there is provided a method of co-expressing a chimeric antigen receptor for example, a chimeric antigen receptor selected from a ‘classical’ or ‘co-stimulatory CAR’ and a glutamine and/or tryptophan transporter comprising the steps of introducing genetic information encoding a suitable CAR with binding specificity to a target or disease antigen and a glutamine and/or tryptophan transporter contained within independent vectors/constructs or within the same construct. The SLC1A5 sequence, an isoform of SLC1A5 sequence or a tryptophan or glutamine transporter may be driven from a separate promoter from that of the CAR to produce two independent mRNAs. The expression of the CAR and the transporter sequence may be achieved by transcription from a common, bi-directional promoter to produce two independent mRNAs. The expression of the CAR and the transporter sequence may be achieved by transcription from a single promoter and incorporation of an IRES between the two coding sequences to produce a single mRNA capable of translating two proteins. The CAR and transporter sequence may be separated by a self-cleaving T2A cleavage sequence providing a single mRNA, driven from a common promoter, translating a single polypeptide which will be co-translationally cleaved to generate two proteins.

Accordingly, a T cell expressing a chimeric antigen receptor and a glutamine and/or tryptophan transporter may be provided.

Suitably there is provided a method to select a T cell which is capable of proliferating in low tryptophan and/or glutamine conditions. The method of selection can comprise the steps of growing the SLC1A5—over-expressing (or alternative over-expressing transporter) T cells in cell culture growth media which contains sub-optimal levels of L-tryptophan, for example, concentrations of L-tryptophan of less than 5 μM. Cells expressing a suitable transporter may also be enriched by propagation in cell culture growth media, which contains sub-optimal levels of L-glutamine, for example, concentrations of L-glutamine of less than 3 μM. Cell culture growth media may also be used which contains sub-optimal levels of both L-tryptophan and L-glutamine. Alternatively, cells expressing the transporter may be enriched by propagation in cell culture growth media which contains the presence of an inhibitor of SLC1A5, such as O-Benzyl-L-Serine, to mimic low tryptophan conditions. Such growth conditions provide a method by which T cells expressing the genetically introduced transporter may be enriched and selected for within the cell culture population, thus selecting against the proliferation of unmodified T cells.

In embodiments, a T cell overexpressing a chimeric antigen receptor and a glutamine or tryptophan transporter may be selected by culturing the cells in medium containing low concentrations of tryptophan and/or low glutamine, or the presence of an inhibitor of SLC1A5, such as O-Benzyl-L-Serine.

In embodiments, an antibody with binding specificity to a glutamine and/or tryptophan transporter can be used to select T cells which are capable of proliferating in low tryptophan and/or glutamine conditions. Suitably, an antibody may be selected from anti-SLC1A5, anti-SLC7A5, anti-LAT1 or anti-SLC3A2.

In embodiments, an antibody directed against a transporter overexpressed on a modified T cell, which is therapeutically administered to a subject, may be separately administered to the subject as a safety mechanism by which to deplete the administered modified T cells in the event of an adverse reaction to the treatment. Such antibodies would instigate antibody dependent cell mediated cytotoxicity (ADCC) to deplete the modified T cells. Such therapeutic antibodies may include but are not limited to anti-SLC1A5, anti-SLC7A5, anti-LAT1 or anti-SLC3A2.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

As used herein, the articles “a” and “an” refer to one or to more than one (for example to at least one) of the grammatical object of the article.

“About” shall generally mean an acceptable degree of error for the quantity measured given the nature or precision of the measurements.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Preferred features and embodiments of each aspect of the invention are as for each of the other aspects mutatis mutandis unless context demands otherwise.

Embodiments of the present invention will now be described by way of example only with reference to the accompanying figures in which:

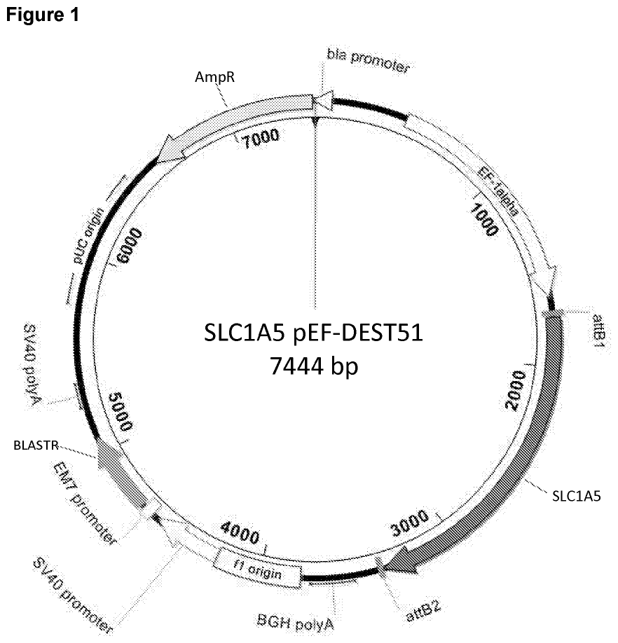

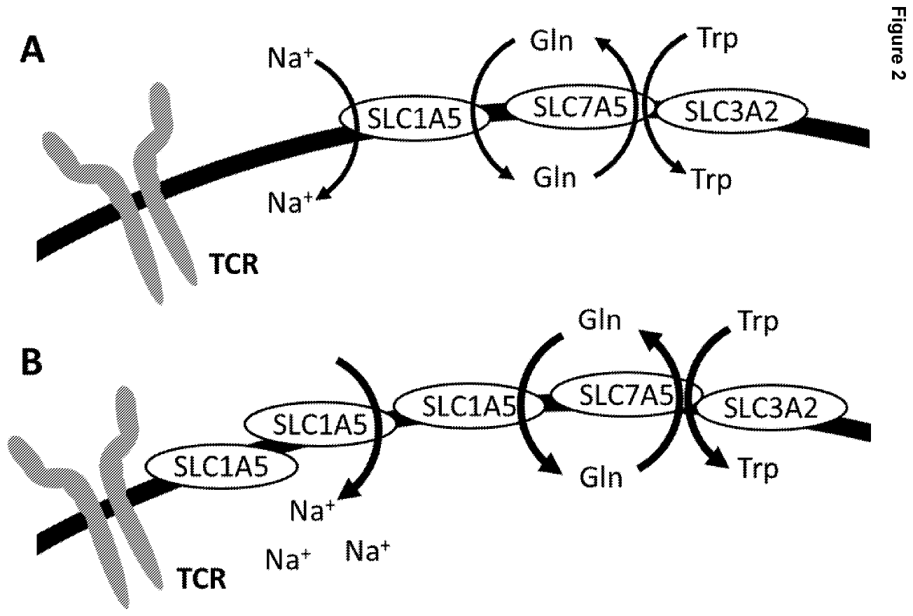

FIG. 1—illustrates a proposed nucleic acid construct of the invention wherein the SLC1A5 gene is expressed under an EF1 alpha promoter with a BGH polyadenylation signal for mRNA stability. The pEF-DEST51 vector (Life Technologies) also contains a pUC origin of replication and the beta-lactamase gene (annotated AmpR) conferring resistance to the antibiotic ampicillin to allow for sufficient propagation of the plasmid DNA in a bacterial host.

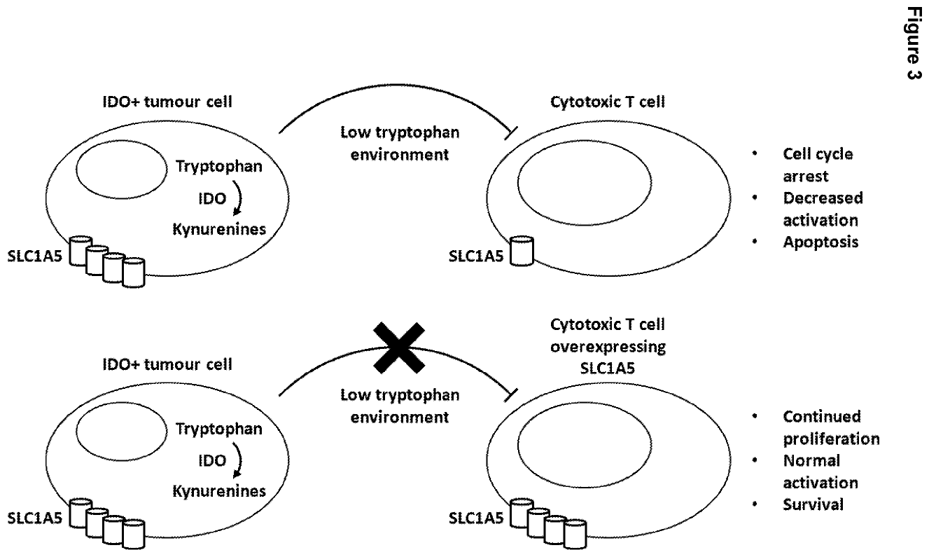

FIG. 2 illustrates A) An unmodified T cell in which SLC1A5 transports glutamine in a sodium dependent manner which provides substrate for SLC7A5/SLC3A2 (LAT1) antiporter complex which is largely dependent on the efflux of intracellular glutamine for the import of amino acids such as tryptophan. B) A modified T cell that is engineered to over-express SLC1A5 by means of transfection or transduction of the T cell with a SLC1A5 containing vector, causing expression of SLC1A5 and thus increased uptake of glutamine into the cell. This provides additional substrate (glutamine) for SLC7A5/SLC3A2 (LAT1) antiporter complex thus increasing the import/uptake of tryptophan.

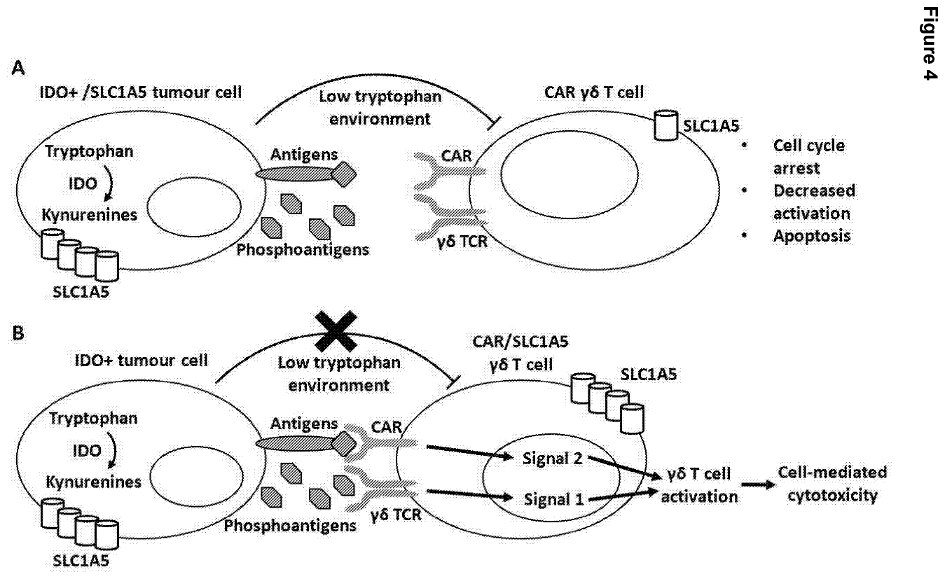

FIG. 3 provides an illustrative example of the proposed mode of action of SLC1A5 overexpressing T cells and illustrates (A) IDO+ tumour cells that create a low tryptophan microenvironment which causes cell cycle arrest, decreased activation and apoptosis in cytotoxic T cells. The tumour cells compensate for the low tryptophan conditions by upregulating expression of SLC1A5. (B) By equipping the T cell with the same mechanism of compensation as the tumour cell via SLC1A5 overexpression, the T cell is able to function in the low tryptophan tumour microenvironment.

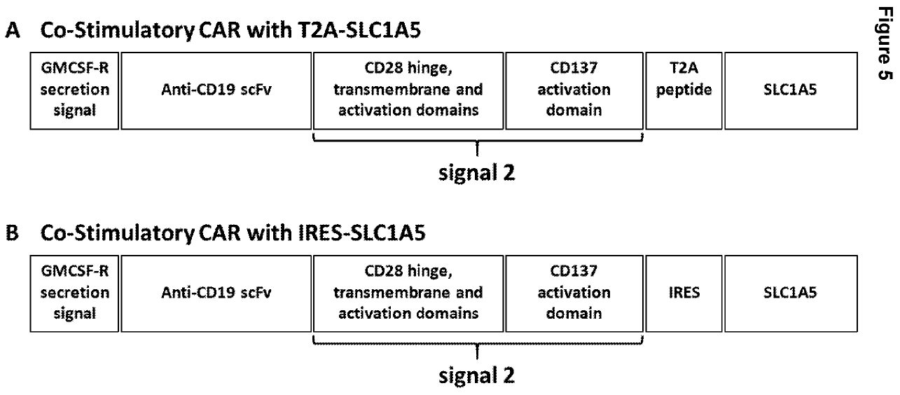

FIG. 4 provides an illustrative example of the proposed mode of action of SLC1A5 and co-stimulatory CAR overexpressing gamma delta T cells and illustrates (A) IDO+ tumour cells create a low tryptophan microenvironment which causes cell cycle arrest, decreased activation and apoptosis in chimeric antigen receptor expressing γδ T cells. The tumour cells compensate for the low tryptophan conditions by upregulating expression of SLC1A5 which allows for greater import of tryptophan. (B) By equipping the gamma delta CAR-T cell with the same mechanism of compensation as the tumour cell via SLC1A5 overexpression, the gamma delta CAR-T cell is able to function in the low tryptophan tumour microenvironment, and elicit full cytotoxic effector function by recognising phosphoantigens via the γδ TCR and the disease antigen via the CAR (or co-stimulatory CAR); the CAR/SLC1A5 γδ T cell is able to function and perform cell-mediate cytotoxicity.

FIG. 5. illustrates the co-stimulatory CAR construct comprising the GMCSF-R secretion signal domain, scFv against CD19, CD28 hinge, transmembrane and activation domains and CD137 (4-1BB) activation domain. SLC1A5 co-expression from the same construct may be achieved by either a C-terminal T2A self-cleaving peptide or an internal ribosomal entry site (IRES) before the SLC1A5 sequence.

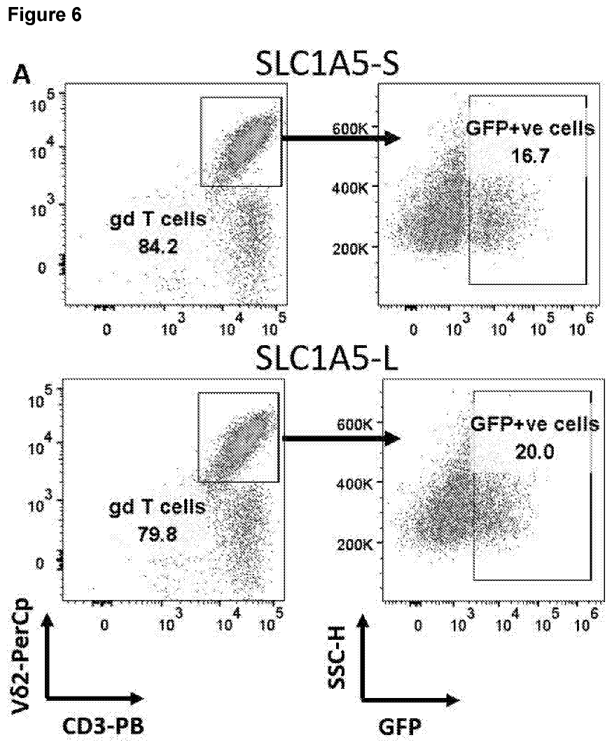

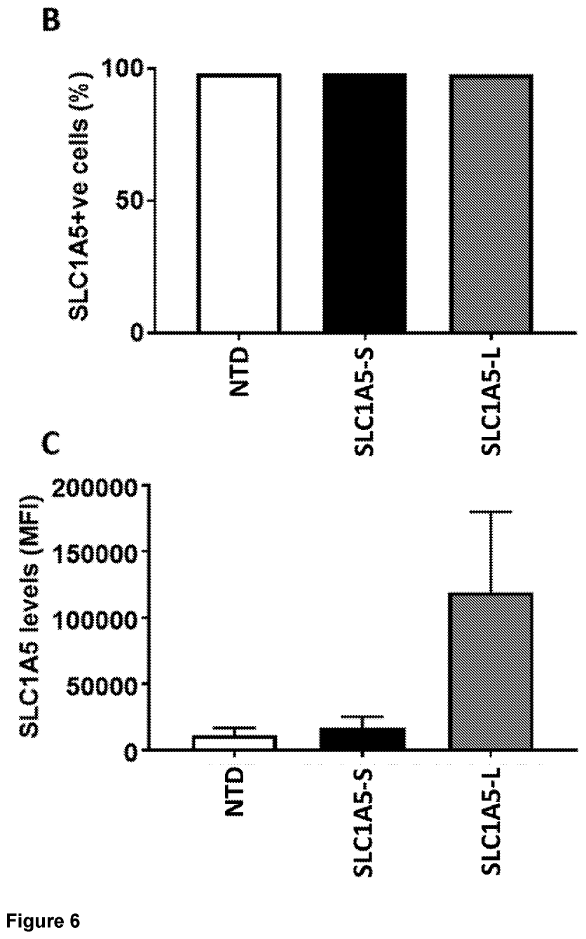

FIG. 6 illustrates the transduction efficiency and the expression levels of SLC1A5 in Vdelta2 γδ T cells. PBMCs were transduced with lentiviral vectors carrying SLC1A5-L (accession #NP_005619.1) or SLC1A5-S (accession #NP_001138616.1) and GFP sequences, 48 hrs after their stimulation with zoledronic acid. Transduced cells were expanded for a further 16 days and percentage of GFP-positive cells was measured by flow cytometry. Transduction efficiency measured by GFP-positive cells (%) was 16.7% (SLC1A5-S) and 20% (SLC1A5-L) (A). Cells were also stained intracellularly for SLC1A5 by fixation/permeabilisation and analysed by flow cytometry. At least 97% of γδ T cells expressed SLC1A5, regardless of transduction (C). However, expression levels of SLC1A5 (measured by the mean fluorescence intensity, MFI) were higher in γδ T cells transduced with SLC1A5-L (˜10-fold) or SLC1A5-S (˜1.5-fold) than that in non-transduced γδ T cells (B). These data demonstrate that γδ T cells transduced with SLC1A5-L or SLC1A5-S have higher expression levels of the transporter.

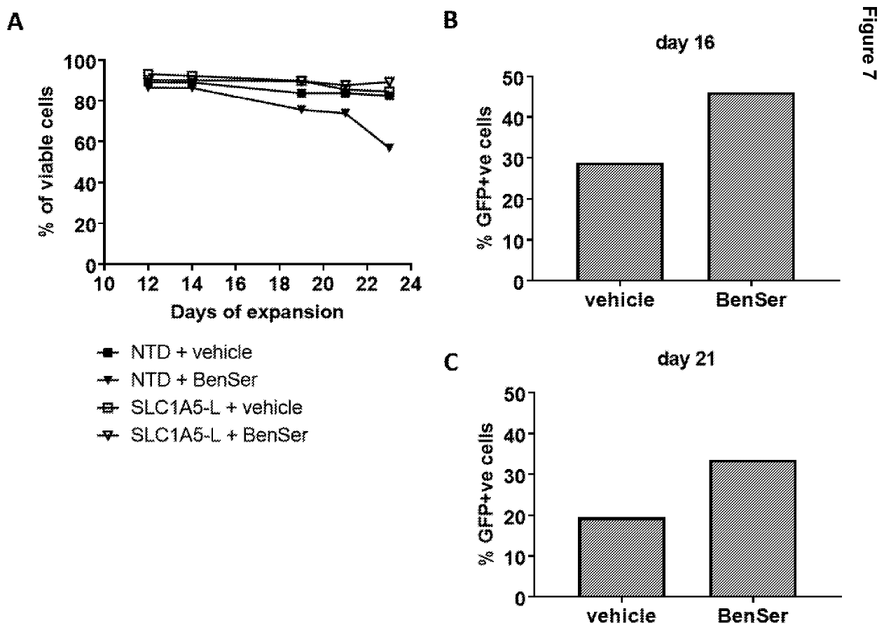

FIG. 7 illustrates the resistance to the SLC1A5 inhibitor O-Benzyl-L-Serine (BenSer) and the resulting positive selection of Vdelta2 γδ T cells transduced with lentivirus containing the SLC1A5-L sequence. PBMCs were transduced, 48 hrs after their stimulation with zoledronic acid. The cells were expanded in ALys medium with or without BenSer for up to 21 days. Cells at two or three weeks of expansion were analysed by flow cytometry measuring viability (using propidium iodide) or GFP-positive γδ T cells, to determine the survival advantage of transduced γδ T cells under selective pressure. After three weeks of expansion, in the presence of BenSer, a reduction of viability was found in untransduced γδ T cells compared to γδ T cells in earlier stages of expansion (from above 80% at day 12 to below 60% at day 23) (A). However, γδ T cells transduced with the SLC1A5-L isoform did not show reduction in viability, becoming resistant to BenSer, (A). Moreover, an increase in GFP-positive γδ T cells was recorded after two or three weeks of expansion in the presence of BenSer compare to the vehicle control (˜17% and ˜14% increase respectively (B and C). Therefore, these demonstrate that overexpression of SLC1A5-L renders γδ T cells resistant to the BenSer in culture medium, which mimics low levels of L-tryptophan.

### EXAMPLES

In order to compensate for a shortage or depletion of tryptophan caused by the expression of IDO and TDO wherein the tumour cells regulate the expression of amino acid transporters including SLC1A5 and its truncated isoforms which in turn enhance uptake of glutamine and tryptophan into the tumour cell and cause a tryptophan depleted micro environment around the tumour cell, the present invention provides T cells which have upregulated expression of such amino acid transporters including SLC1A5 and its truncated isoforms to allow the T cells to proliferate in such low tryptophan concentrations.

In a particular embodiment discussed herein, SLC1A5 can be co-expressed on the same vector as a chimeric antigen vector construct which is expressed by and provided on a T cell.

Suitably, SLC1A5 can be expressed under a promoter or linked to the expression of a chimeric antigen receptor by an internal ribosome entry site (IRES) or a T2A cleavage sequence providing a single mRNA, driven from a common promoter, translating a single polypeptide which will be co-translationally cleaved to generate two proteins (see FIG. 5). As discussed herein, a vector in which the SLC1A5 transporter is provided can be a mammalian expression vector such one from the Gateway ‘DEST’ series (Life Technologies), a lentiviral vector such as from the pCDH suite provided by System Biosciences, a transposon vector or a vector suitable for the generation of minicircles.

The co-expression of SLC1A5 provides several advantages in which:
1. Transfected T cells expressing a chimeric antigen receptor and SLC1A5 have a growth advantage in low tryptophan and/or glutamine conditions and thus these conditions can be used to select for cells expressing both the chimeric antigen receptor and the transporter
2. T cells over expressing SLC1A5 alone or in conjunction with a chimeric antigen receptor are more resistant to proliferative arrest following exposure to low tryptophan conditions caused by tumour expressed IDO or TDO.
3. T cells expressing SLC1A5 alone or in conjunction with a chimeric antigen receptor may be selectively depleted following adoptive cell transfer by use of an antibody specific for the SLC1A5 transporter

As discussed herein, T cells which have a growth advantage in low tryptophan and/or glutamine conditions can be selected following transfection using low conditions of glutamine and/or tryptophan in the cell culture media.

Alternatively, suitable T cells can be positively selected using, for example, antibodies able to bind to SLC1A5 or the like. For example, using magnetic activated cell sorting (MACS) technologies, fluorescent-activated cells sorting (FACS) or similar techniques known to those skilled in the art.

### Example 1

Generation of a Vector to Allow Transfection of a T Cell

DNA encoding the SLC1A5 long isoform was obtained from GeneArt (Life Technologies) in the pDONR221 backbone between attL1 and attL2 recombination sites. The SLC1A5 can then be recombined into a variety of Gateway compatible destination vectors using the LR Clonase II recombinase reaction (Life Technologies). In this example the SLC1A5 was recombined into pEF-DEST51 containing an EF1 alpha promoter and a BGH polyadenylation signal (see FIG. 1).

### Example 2

Transfection of a T Cell to Provide for Expression of SLC1A5 at a Level Which Allows the T Cell to Overcome T Cell Proliferative Arrest Due to Tryptophan Concentrations Being Decreased, for Example, Decreased Below 5 μM.

T cells are electroporated with the vector described in example 1 by either Nucleofection (Lonza) or the Neon electroporation system (Thermo Fisher). Following recovery in complete medium (such as IMDM) for 24 to 48 hours, T cells are cultured in IMDM media containing less than 5 μM L-trytophan.

### Example 3

Example of Providing the SLC1A5 Long Isoform Gene Using a Lentivirus System.

The SLC1A5 long isoform gene in example 1 was cloned into the pCDH vector backbone (Systems Bioscience). Lentiviral supernatants were generated by co-transfecting HEK293T cells with the pCDH vector and a mix of lentiviral packaging vectors expressing the gag, pol, rev and env genes necessary for viral production using Purefection transfection reagent (System Bioscience). Viral supernatants were collected at 48 and 72 hours post-transfection and concentrated using PEG-it (System Bioscience). T cells were plated and infected by addition of the lentivirus.

The SLC1A5 long isoform (SLC1A5-L) and its truncated isoform (SLC1A5-S) were transduced in γδ T cells by a lentivirus system containing either SLC1A5-L or SLC1A5-S sequences, followed by T2A and GFP sequences The functional transduction efficiency was 16.7 (SLC1A5-S) and 20% (SLC1A5-L), based on GFP-positive γδ T cells (see FIG. 6A). Moreover the vast majority of γδ T cells expressed SLC1A5, whether they were transduced or not (see FIG. 6B). However, expression levels of SLC1A5 were 10-fold (SLC1A5-L) or 1.5-fold higher (SLC1A5-S) in transduced γδ T cells than that in non-transduced γδ T cells (see FIG. 6C).

### Example 4

Example of Providing an SLC1A5 Long Isoform Gene Using a Transposon Based System.

The SLC1A5 long isoform gene in example 1 was cloned into the PB51x vector (System Bioscience). T cells were co-transfected with the PB51x vector and the ‘Super’ PiggyBac transposase expression vector (System Bioscience) by either Nucleofection (Lonza) or the Neon electroporation system (Thermo Fisher).

### Example 5

Use of SLC1A5 as a Selection Marker and Discussion of Media Which Could be Used to Allow a Selective Pressure Environment.

T cells are either transfected (as in examples 2 and 4) or transduced (as in example 3) with a SLC1A5 expressing construct (such as in example 1) allowing the overexpression of SLC1A5. Following transfection/transduction of the T cells with a vector capable of expressing SLC1A5, the T cells are allowed a recovery period of typically 24 to 48 hours in complete media such as ALyS or IMDM. After the recovery period the T cells are cultured in ALyS or IMDM media containing less than 5 μM L-tryptophan or containing less than 4 mM L-glutamine or a combination of conditions Growth is monitored in comparison to unmodified T cells.

As the actual concentration of L-tryptophan in cell culture systems was not controlled over time, this can impact on the response of gamma delta T cells. Therefore, the inhibitor of SLC1A5 O-Benzyl-L-Serine (BenSer) was added into the culture medium, to generate a controlled selective pressure environment, which mimics the low L-tryptophan condition. PBMCs transduced with lentiviral vectors carrying SLC1A5-S or SLC1A5-S (48 hrs after stimulation with zoledronic acid) were cultured in ALys medium with or without BenSer. During the second and third week of expansion, γδ T cells were analysed by flow cytometry to measure cell viability and GFP-positive cells, to determine the survival advantage of transduced γδ T cells under selective pressure. After three weeks of expansion, the presence of BenSer reduced the viability of untransduced γδ T cells, compared to earlier stages of expansion (FIG. 7A). However, γδ T cells transduced with the SLC1A5-L isoform did not show reduction in viability, and therefore became resistant to BenSer, (FIG. 7A). Moreover, the percentage of GFP-positive γδ T cells increased after two and three weeks of expansion in the presence of BenSer compare to the vehicle control (approximately 17% and 14% increase respectively, FIG. 7B-C). Therefore, the overexpression of the SLC1A5-L isoform renders gamma delta T cells resistant to the BenSer in culture medium, which mimics low levels of L-tryptophan.

Although the invention has been particularly shown and described with reference to particular examples, it will be understood by those skilled in the art that various changes in the form and details may be made therein without departing from the scope of the present invention.

1.-26. (canceled)

27. A method of selecting a modified T cell adapted to overexpress SLC1A5, an isoform of SLC1A5 or an alternative tryptophan or glutamine transporter, wherein the method comprises the steps:
a. culturing a population of T cells comprising modified T cells adapted to overexpress SLC1A5, an isoform of SLC1A5 or an alternative tryptophan or glutamine transporter in a media with at least one of L-tryptophan at a concentration of less than 5 μM and L-glutamine at a concentration of less than 3 μM, or the presence of an inhibitor of SLC1A5, and
b. selecting those modified T cells which proliferate when cultured according to step a.

28. The method of selecting a modified T cell adapted to overexpress SLC1A5, an isoform of SLC1A5 or an alternative tryptophan or glutamine transporter, as claimed in claims 27, wherein the method further comprises the steps:
c. providing a binding member with binding specificity to at least one of SLC1A5, an isoform of SLC1A5 or an alternative tryptophan or glutamine transporter, to a cell expressing at least one of SLC1A5, an isoform of SLC1A5 or an alternative tryptophan or glutamine transporter,
d. optionally, detecting the binding of the binding member to the cell in step a, and
e. selecting the cell to which the binding member is bound.

29. The method of selecting a modified T cell adapted to overexpress SLC1A5, an isoform of SLC1A5 or an alternative tryptophan or glutamine transporter as claimed in claim 28 wherein the method further comprises the steps:
f. isolating a cell to which the binding member is bound.

30. The method of selecting a modified T cell of claim 27 wherein the inhibitor of SLC1A5 is O-Benzyl-L-Serine.

31. The method of any of claims 27 to 30 wherein the modified T cell co-expresses a chimeric antigen receptor and a glutamine and/or tryptophan transporter provided by the same construct.

32. A modified T cell provided by the method of claim 31.

33. The modified T cell of claim 31 wherein the T cell is adapted to express SLC1A5, an isoform of SLC1A5 or a tryptophan or glutamine transporter at a level at least twice the expression level observed in an unmodified activated T cell.

34. The modified T cell of claim 32 or 33 wherein the T cell expresses a gamma delta T cell receptor and a co-stimulatory chimeric antigen receptor (CAR) wherein the costimulatory CAR comprises, an antigen binding domain, a transmembrane domain and an intracellular signalling domain wherein the intracellular signalling domain provides a co-stimulatory signal (signal 2 only) to the T cell following binding of antigen to the extracellular antigen binding domain.

35. The modified T cell of claim 32 or 33 wherein the T cell expresses a T cell receptor and a chimeric antigen receptor (CAR) wherein the CAR comprises, an antigen binding domain, a transmembrane domain and an intracellular signalling domain wherein the intracellular signalling domain provides a signal 1 response only, for example from a CD3zeta domain, to the T cell following binding of antigen to the antigen binding domain.

36. The modified T cell of claim 32 or 33 wherein the T cell expresses a T cell receptor and a chimeric antigen receptor (CAR) wherein the CAR comprises, an antigen binding domain, a transmembrane domain and an intracellular signalling domain wherein the intracellular signalling domain provides a signal 1 response, for example from a CD3zeta domain, and a signal 2 response from a co-stimulatory domain to the T cell following binding of antigen to the antigen binding domain.

37. The modified T cell of claim 34 wherein the T cell expresses a gamma delta T cell receptor and a chimeric antigen receptor (CAR) wherein, in use, signal 1 is provided by a first binding event of the TCR on the gamma delta T cell to the cell binding target recognised by the TCR and signal 2 is provided by a second binding event of antigen to the antigen binding domain of the co-stimulatory CAR and in combination signal 1 and signal 2 from both first and second binding events respectively activate the T cell.

38. The modified T cell of any one of claims 32 to 37 wherein the T cell expresses a gamma delta T cell receptor wherein the gamma delta (γδ) T cell is of the Vγ9Vδ2 subtype.

39. The modified T cell of any of claims 32 to 38 wherein SLC1A5 is overexpressed in conjunction with SLC7A5 and SLC3A2 to form a LAT1 transporter.

40. The method of any of claims 27 to 31 and the modified T cell of any of claims 32 to 39 wherein the isolated nucleic acid encoding the transporter is selected from a high-affinity glutamate and neutral amino acid transporter family (SLC1A1, SLC1A2, SLC1A3, SLC1A4, SLC1A5, SLC1A6, SLC1A7); heavy subunits of heterodimeric amino acid transporters (SLC3A1, SLC3A2); a member of the sodium- and chloride-dependent sodium:neurotransmitter symporter family (SLC6A1, SLC6A2, SLC6A3, SLC6A4, SLC6A5, SLC6A6, SLC6A7, SLC6A8, SLC6A9, SLC6A10, SLC6A11, SLC6A12, SLC6A13, SLC6A14, SLC6A15, SLC6A16, SLC6A17, SLC6A18, SLC6A19, SLC6A20) or a member of a cationic amino acid transporter/glycoprotein-associated family (SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14).

41. A method of treating a cancer, the method comprising administration of an effective amount of a modified T cell of any one of claims 32 to 40 to a subject in need thereof.

42. A modified T cell of any one of claims 32 to 40 for use in medicine.

43. A modified T cell of any one of claims 32 to 40 for use in the treatment of cancer or a virus.

44. The method of claim 41 wherein the T cell is isolated from a subject with a disease to be treated.

45. A pharmaceutical composition comprising a modified T cell of any one of claims 32 to 40 and a therapeutic agent.

46. Use of a modified T cell according to any one of claims 32 to 40 in the manufacture of a medicament for treating or preventing disease.

47. Use of a modified T cell in the manufacture of a medicament according to claim 46 wherein the disease is cancer.

48. A method of treating a cancer as claimed in claim 41 wherein the, the method comprises administering at a first time point an effective amount of a modified T cell of any one of claims 32 to 40 to a subject in need thereof, wherein the method further comprises a step of administering to the subject at a second later time point a binding member with binding specificity to SLC1A5, an isoform of SLC1A5 or an alternative tryptophan or glutamine transporter capable of binding to the modified T cell to selectively bind to and reduce the modified T cells present in the subject.
